(19) United States
(12) Patent Application Publication
(10) Pub. No.: US 2026/0354782 A1
(43) Pub. Date: Nov. 12, 2026

(54) LARGE SUBJECT ROBOTIC RADIOGRAPHIC SYSTEM AND METHOD

(71) Applicant: UNIVERSITY OF FLORIDA RESEARCH FOUNDATION, INCORPORATED, Gainesville, FL (US)

(72) Inventors: Scott Arthur BANKS, Gainesville, FL (US); Adam Henry BIEDRZYCKI, Gainesville, FL (US); Frank J. Bova, Gainesville, FL (US); Shannon RIDGEWAY, Gainesville, FL (US)

(21) Appl. No.: 19/475,041

(22) PCT Filed: Apr. 15, 2024
(86) PCT No.: PCT/US2024/024609

§ 371 Date: Oct. 13, 2025

Related U.S. Application Data
(60) Provisional application No. 63/459,000, filed on Apr. 13, 2023.

(51) Int. Cl. A61B 6/50; A61B 6/00; A61B 6/40; A61B 6/42; A61B 34/10; A61B 34/20

(52) U.S. Cl. CPC A61B 6/508; A61B 6/4028; A61B 6/4078; A61B 6/4233; A61B 6/4452; A61B 6/56; A61B 34/10; A61B 34/20; A61B 2034/105

(57) ABSTRACT

A system and method are provided for capturing image data of a large subject. The system includes a radiation source configured to generate a radiation fan beam in two dimensions and a detector configured to detect the radiation fan beam. The system also includes a first support and a second support configured to translate and rotate the radiation source and the detector. The method includes moving the radiation source and the detector, with the first and second supports, to each of a plurality of incremental positions along an anatomical region of the large subject. The method further includes capturing image data at each incremental position with the radiation source and the detector. The method further includes moving the radiation source and detector, with the first and second supports, to offset relative motion between the large subject and the radiation source or detector.

### CROSS-REFERENCE TO RELATED APPLICATIONS

This application claims benefit of U.S. Provisional Application No. 63/459,000, filed Apr. 13, 2023, under 35 U.S.C. § 119(e) and 35 U.S.C. § 120.

### BACKGROUND

Radiographic systems are used to capture image data (e.g. x-ray) of a subject, for purposes of evaluating anatomical regions of the subject for various applications. These applications include diagnosis of medical conditions or planning medical treatments and interventions to treat any diagnosed medical conditions.

### SUMMARY

Conventional radiographic systems are known which are used to capture image data of a subject. For example, U.S. Pat. No. 7,441,953 ('953 patent hereafter), which is assigned to the same assignee as the present invention, discloses such a radiographic system. The system discloses position markers placed on a human subject which are used to track movement of a small joint (e.g. ankle/foot) through various movement phases. An x-ray source is provided that generates an x-ray cone beam which is directed through a cross-sectional area of the joint and is detected by the detector. Based on the tracking data, the system adjusts the position of the x-ray source and the detector to reduce relative movement between the joint and the x-ray source or detector and ensure that the captured image data at each movement phase remains unblurred.

In the present invention, the inventors designed a radiographic system which is used to capture image data of a large subject (e.g. horse, cow, etc.) having thick anatomical regions to be imaged. While the inventors recognized that their '953 patent disclosed a radiographic system, they realized that the x-ray cone beam of the '953 patent could not be used to capture image data of the thick anatomical regions of the large subject. This is due to excess scattering radiation that would be picked up by the detector if the x-ray cone beam was transmitted through these thick anatomical regions of the large subject. Hence, to address this issue the inventors decided to use a line scanning x-ray source which generates a two-dimensional (2D) x-ray fan beam in a single plane. The inventors reasoned that the 2D x-ray fan beam would not cause excess scattering through the thick anatomical regions of the large subject and thus would generate image data with sufficient signal to noise ratio.

Additionally, in designing the radiographic system herein, the inventors recognized that the x-ray source and the detector would need the capability of moving over relatively large space in order to encompass the dimensions of the large subject. The '953 patent disclosed a robotic arm with a base and rotational degrees of freedom to mount the x-ray source and the detector, so that the x-ray source and detector could be rotated about the small joint (e.g. ankle/foot). However, the inventors recognized that this robotic arm structure would not be effective in the radiographic system for the large subject as it would require a prohibitively large volume in order to facilitate rotation of the robotic arm over the dimensions of the large subject. Hence, to address this issue the inventors developed the radiographic system herein with an x-ray source and detector that feature both translational and rotational degrees of freedom. The inventors recognized that this support structure would facilitate housing the system in a much more compact volume than a design based on the '953 patent.

In a first embodiment of the present invention, a system is provided that includes a radiation source configured to generate a radiation fan beam in a two-dimensional (2D) plane. The system also includes a detector spaced apart from the radiation source and configured to detect the radiation fan beam in the 2D plane. The system also includes a first support configured to mount the radiation source. The first support is configured to translate the radiation source along a first axis and further configured to rotate the radiation source relative to the first axis. The system further includes a second support configured to mount the detector. The second support is configured to translate the detector along a second axis and further configured to rotate the radiation source relative to the second axis. The system further includes a position marker coupled to each of the radiation source, the detector and a subject positioned between the radiation source and the detector. The position markers are configured to measure a position of each of the radiation source, the detector and the subject. The system also includes a processor and a memory including one or more sequences of instructions. The memory and the sequences of instructions are configured to, with the processor, cause the system to transmit a first signal to the first support and the second support to cause the radiation source and the detector to move to each of a plurality of incremental positions along an anatomical region of the subject. The memory and the sequences of instructions are further configured to, with the processor, cause the system to receive motion data, at each incremental position, from the position marker coupled to each of the radiation source, the detector and the subject to detect relative motion between the subject and the radiation source or the detector. The memory and the sequences of instructions are further configured to, with the processor, cause the system to transmit a second signal to the first support and the second support, at each incremental position, to move the radiation source and the detector to offset the detected relative motion. The memory and the sequences of instructions are further configured to, with the processor, cause the system to transmit a third signal to the radiation source to cause the radiation source to generate the radiation fan beam in the 2D plane at each incremental position along the anatomical region of the subject. The memory and the sequences of instructions are further configured to, with the processor, cause the system to receive image data from the detector based on the detection of the radiation beam in the 2D plane at each incremental position. The memory and the sequences of instructions are further configured to, with the processor, cause the system to store, in the memory, the image data at each incremental position.

In a second embodiment of the present invention, a method is provided that includes transmitting, from a processor, a first signal to a first support and a second support to cause a radiation source and a detector to move to each of a plurality of incremental positions along an anatomical region of a subject. The method further includes receiving, from position markers attached to each of the radiation source, the detector and the subject, motion data to detect relative motion between the subject and the radiation source or the detector at each incremental position. The method further includes transmitting, from the processor, a second signal to the first support and the second support to move the radiation source and the detector to offset the detected relative motion at each incremental position. The method further includes transmitting, from the processor, a third signal to the radiation source to cause the radiation source to generate a radiation fan beam in a two-dimensional (2D) plane at each incremental position. The method further includes receiving, from the detector, image data based on detection of the radiation fan beam in the 2D plane at each incremental position. The method further includes storing, in a memory, the image data at each incremental position.

In yet another embodiment of the present invention, the method further includes providing a guided instrument with a position marker. The method further includes combining, with the processor, the stored image data at one or more incremental positions of the anatomical region into a 3D model of the anatomical region of the subject. The method further includes outputting, on a display, the 3D model of the anatomical region of the subject. The method further includes varying, with an input device, an orientation of the 3D model on the display to a desired orientation. The method further includes receiving, at the processor, data from the position marker of the guided instrument. The method further includes outputting, on the display, a virtual guided instrument in the desired orientation of the 3D model based on the received data from the position marker of the guided instrument. The method further includes moving the guided instrument to perform a treatment of the anatomical region of the subject based on viewing the virtual guided instrument moving relative to the desired orientation of the 3D model on the display.

In yet another embodiment of the present invention, the method further includes determining, with the processor, a rate of energy absorption of the radiation fan beam in the 2D plane in the anatomical region based on the received image data at each incremental position. The method further includes determining, with the processor, a ratio of one or more tissue types within the anatomical region of the subject based on the determined rate of absorption. The method further includes outputting, on a display, data that indicates the determined ratio of the one or more tissue types within the anatomical region of the subject.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a block diagram that illustrates an example of a top view of a radiographic system for large subjects, according to an embodiment;

FIG. 1B is an image that illustrates an example of a front perspective view of the radiographic system of FIG. 1A, according to an embodiment;

FIG. 1C is an image that illustrates an example of a rear view of the radiographic system of FIG. 1A, according to an embodiment;

FIG. 1D is an image that illustrates an example of a rear view of the radiographic system of FIG. 1A, according to an embodiment;

FIG. 2A is an image that illustrates an example of image data with motion blur captured with a conventional radiographic system;

FIG. 2B is an image that illustrates an example of image data without motion blur captured with the radiographic system of FIG. 1A, according to an embodiment;

FIG. 3A is an image that illustrates an example of image data captured with a conventional radiographic system having a dynamic intensity range outside a desired range;

FIG. 3B is an image that illustrates an example of image data captured with the radiographic system of FIG. 1A having dynamic intensity range within a desired range, according to an embodiment;

FIG. 3C is an image that illustrates an example of image data captured with a conventional radiographic system having a low signal to noise ratio (SNR);

FIG. 3D is an image that illustrates an example of image data captured with the radiographic system of FIG. 1A having a high SNR, according to an embodiment;

FIG. 4 is an image that illustrates an example of a front view of a display used while performing a treatment of an anatomical region of the large subject, according to an embodiment;

FIG. SA is a flow diagram that illustrates an example method to capture image data with the radiographic system of FIG. 1A, according to an embodiment;

FIG. 5B is a flow diagram that illustrates an example method to perform treatment on a large subject with captured image data from the radiographic system of FIG. 1A, according to an embodiment;

FIG. 5C is a flow diagram that illustrates an example method to measure a ratio of one or more tissue types in an anatomical region of a large subject with captured image data from the radiographic system of FIG. 1A, according to an embodiment;

FIG. 6 is a block diagram that illustrates a computer system upon which an embodiment of the invention may be implemented; and

FIG. 7 illustrates a chip set upon which an embodiment of the invention may be implemented.

### DETAILED DESCRIPTION

A method and apparatus are described for using a radiographic system to capture image data of anatomical regions of a large subject. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive-only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of using a radiographic system to capture image data of anatomical regions of a large subject (e.g. horse, cow, etc.). However, the invention is not limited to this context. In other embodiments, the invention is described in the context of using image data of anatomical regions of a large subject (e.g. horse, cow, etc.) to perform treatments to the anatomical regions. In an example embodiment, the treatment is performed on the anatomical regions of the large subject without applying anesthesia. For purposes of this description, “large subject” means any large animal or mammal, whether aquatic or land based. In one example, the large subject can include land based animals or mammals including but not limited to dogs, horses, giraffes, elephants, etc. and other similar sized animals and mammals. In another example, the large subject can include aquatic or water based animals or mammals including manatees, dolphins, whales, etc. and other similar sized water based animals and mammals. In other embodiments, “large subject” includes human subjects (e.g. standing human subject). Although “large subject” is used to classify the subject used in the invention, there is no minimum size threshold for the subjects used in the invention. In yet another example embodiment, the treatment is performed on the anatomical regions of the large subject while the large subject remains standing. In still other embodiments, the invention is described in the context of using image data of anatomical regions of the large subject to measure a ratio of one or more tissue types within the anatomical region of the large subject. In an example embodiment, the ratio of the one or more tissue types are measured anti-mortem. In this example embodiment, the ratio difference between one or more of bone tissue, soft tissue, fat tissue, etc. is measured. In one example embodiment, the fat or muscle content is determined in regions of beef, lamb or pork, among other meats. This determined fat or muscle content is used to determine antemortem lean percentage of the meat and marbling.

### 1. System Overview

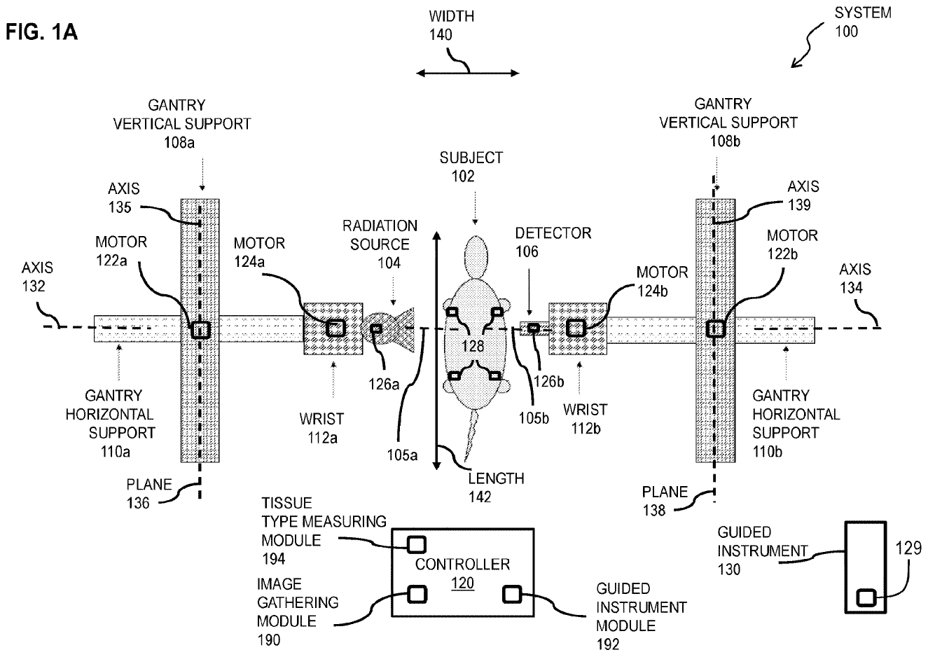

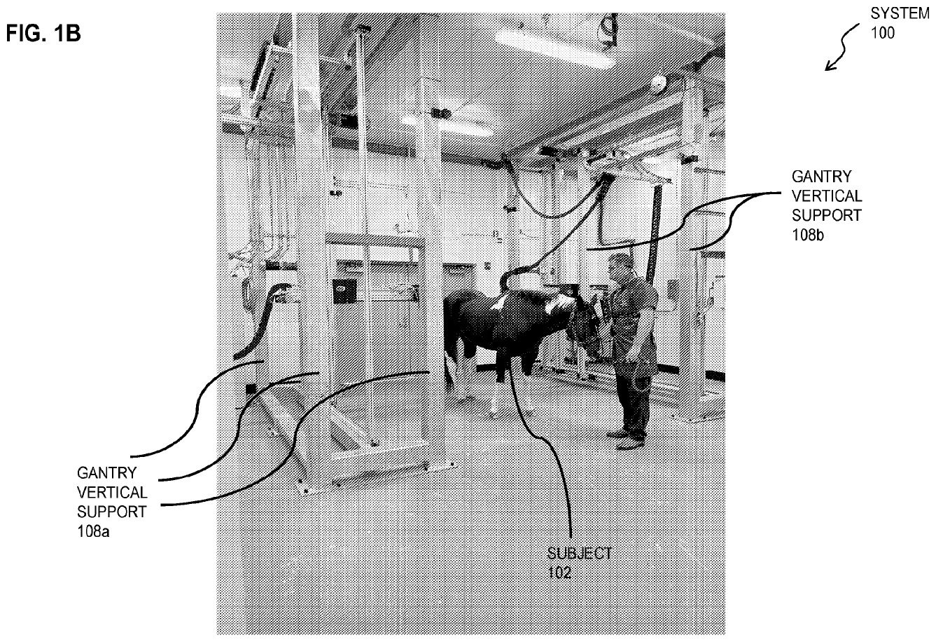

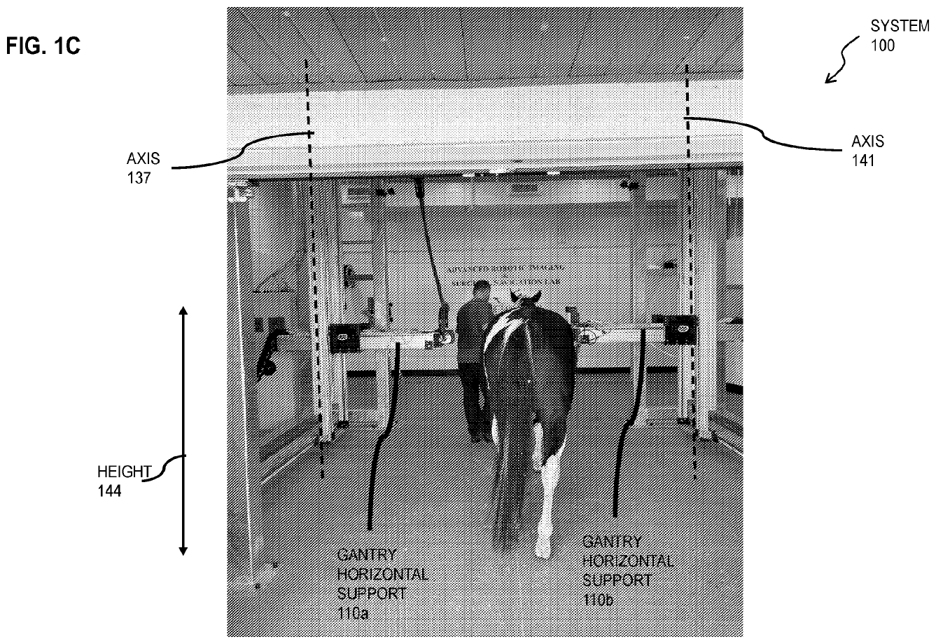

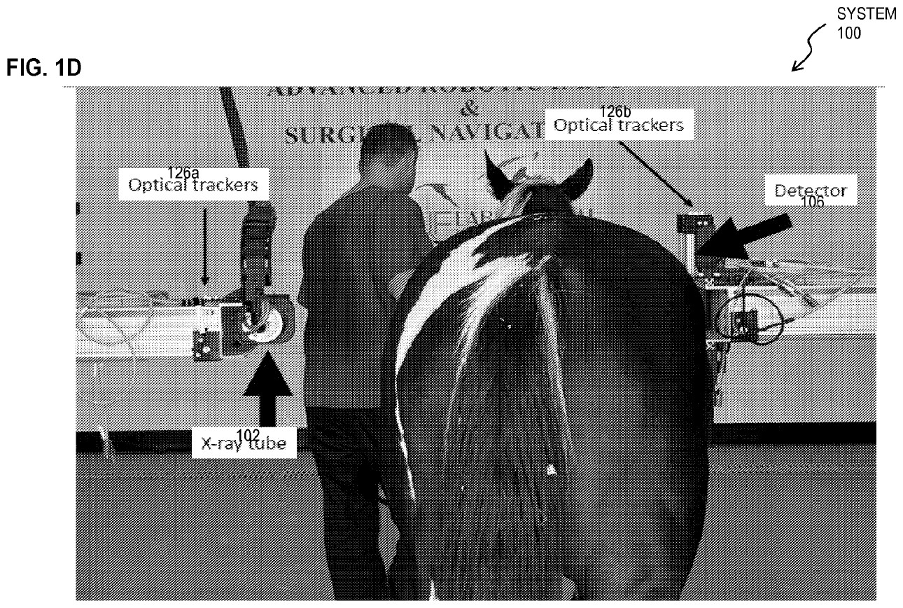

An embodiment of a radiographic system that is used to capture image data of a large subject (e.g. horse, cow, etc.) will now be discussed. FIG. 1A is a block diagram that illustrates an example of a top view of a radiographic system 100 for a large subject 102, according to an embodiment. FIGS. 1B through 1D are images that illustrate an example of various views of the radiographic system 100 of FIG. 1A, according to an embodiment.

The radiation source and the detector of the system 100 will now be discussed. As shown in FIG. 1A, in an embodiment the system 100 includes a radiation source 104 configured to generate a radiation fan beam 105a in a two-dimensional (2D) plane. As further shown in FIG. 1B, in an embodiment the system 100 also includes a detector 106 spaced apart from the radiation source 104 and configured to detect the radiation fan beam 105b in the 2D plane after transmission through the large subject 102. In one embodiment, the radiation source 104 is an x-ray tube and the radiation fan beam 105a is an x-ray fan beam in the 2D plane. In this embodiment, the detector 106 is an x-ray detector including a linear array of detectors configured to detect the x-ray fan beam in the 2D plane. In other embodiments, other radiation sources (e.g. CT, MRI, etc.) can be used. In some embodiments, an adjustable collimator is placed at a tube aperture of the radiation source 104 to provide different shapes of the radiation beam (e.g. fan, cone, rectangular, etc.). The inventors recognized that the transmitted radiation fan beam 105a from the radiation source 104 and detected radiation fan beam 105b at the detector 106 advantageously minimize scattering through the relatively large dimension (e.g. width 140) of the large subject 102. As appreciated by one of ordinary skill in the art, the ratio of scattered radiation to transmitted radiation at the detector 106 increases with the large dimension (e.g. width 140) of the large subject 102.

Supports that are used to mount and position the radiation source 104 and detector 106 will now be discussed. As shown in FIG. 1A, in an embodiment a first support is provided to mount the radiation source 104 and is configured to translate the radiation source 104 along one or more axes and is further configured to rotate the radiation source 104 relative to the one or more axes. The translational and/or rotation of the radiation source 104 and detector 106 are performed in order to capture image data from different regions of the subject 102 and/or to offset undesired relative motion between the subject 102 and the radiation source 104 or the detector 106. The inventors recognized that providing both translational and rotational degrees of freedom permitted the system 100 to be housed in a much smaller volume than if only rotational degrees of freedom were employed.

In one embodiment, as shown in FIG. 1A the first support includes a first gantry horizontal support 110a configured to translate the radiation source 104 along a first axis 132 to vary the spacing between the radiation source 104 and the detector 106. In this embodiment, as shown in FIG. 1A, the first support also includes a first gantry vertical support 108a configured to translate the radiation source 104 in a first plane 136 that is orthogonal to the first axis 132. In an example embodiment, the first gantry vertical support 108a is configured to translate the radiation source 104 along an axis 135 within the first plane 136 in order to translate the radiation source 104 along a length 142 of the large subject 102. In another example embodiment, the first gantry vertical support 108a is configured to translate the radiation source 104 along an axis 137 (FIG. 1C) that is orthogonal to the axis 135 within the first plane 136 in order to translate the radiation source 104 along a height 144 of the large subject 102. As further shown in FIG. 1A, in an embodiment a motor 122a is provided that is configured to translate the first gantry horizontal support 110a (and the radiation source 104) along the first axis 132. In this embodiment, the motor 122a is also configured to translate the first gantry vertical support 108a (and the radiation source 104) along either of the axis 135 or the axis 137. In this embodiment, the motor 122a is communicatively coupled with a controller 120 and is configured to translate the radiation source 104 along any of the axes 132, 135, 137 based on one or more signals received from the controller 120.

In one embodiment, as shown in FIG. 1A the second support includes a second gantry horizontal support 110b configured to translate the detector 106 along a second axis 134 to vary the spacing between the radiation source 104 and the detector 106. In this embodiment, as shown in FIG. 1A, the second support also includes a second gantry vertical support 108b configured to translate the detector 106 in a second plane 138 that is orthogonal to the second axis 134. In an example embodiment, the second gantry vertical support 108b is configured to translate the detector 106 along an axis 139 within the second plane 138 in order to translate the detector 106 along the length 142 of the large subject 102. In another example embodiment, the second gantry vertical support 108b is configured to translate the detector 106 along an axis 141 (FIG. 1C) that is orthogonal to the axis 139 within the second plane 138 in order to translate the detector 106 along the height 144 of the large subject 102. As further shown in FIG. 1A, in an embodiment a motor 122b is provided that is configured to translate the second gantry horizontal support 110b (and the detector 106) along the second axis 134. In this embodiment, the motor 122b is also configured to translate the second gantry vertical support 108b (and the detector 106) along either of the axis 139 or the axis 141. In this embodiment, the motor 122b is communicatively coupled with the controller 120 and is configured to translate the detector 106 along any of the axes 134, 139, 141 based on one or more signals received from the controller 120.

The scale at which the first and second supports can translate the radiation source 104 and the detector 106 are now discussed. The inventors of the present invention recognized the importance of designing the first and second supports of the system 100 such that they could translate the radiation source 104 and detector 106 at a dimensional scale that can encompass one or more dimensions of the large subject 102. In an embodiment, the first and second gantry vertical supports 108a, 10b are configured to translate the radiation source 104 and detector 106 in the respective planes 136, 138 to at least encompass the length 142 and height 144 of the large subject 102. As previously discussed, “large subject” herein refers to a range of land animals or mammals (e.g. dogs, horses, cows, etc.) including human subjects as well as aquatic animals or mammals (e.g. dolphins, whales, manatees, etc.) and thus the length 142 and height 144 values will vary based on the category of the large subject.

The rotational degrees of freedom provided by the first and second supports will now be discussed, which advantageously permit the radiation source 104 and detector 106 to be rotated relative to the translational axes. As illustrated in FIG. 1A, in an embodiment, the system 100 includes a first wrist 112a configured to mount the radiation source 104 to the first gantry horizontal support 110a. In this embodiment, the first wrist 112a is configured to rotate the radiation source 104 relative to the first axis 132. Additionally, as illustrated in FIG. 1A, in an embodiment the system 100 includes a second wrist 112b configured to mount the detector 106 to the second gantry horizontal support 110b. In this embodiment, the second wrist 112b is configured to rotate the detector 106 relative to the second axis 134. In one embodiment, the system 100 also includes respective motors 124a, 124b at the wrists 112a, 112b that are configured to rotate the respective radiation source 104 and detector 106 relative to the respective axes 132, 134 based on one or more signals received from the controller 120.

Position markers that are used to track the position of the subject 102 and one or more components of the system 100 will now be discussed. As shown in FIG. 1A, in one embodiment the system 100 includes position markers 126a, 126 attached to each of the radiation source 104 and the detector 106. In another embodiment, the system 100 includes position markers 128 attached to the large subject 102. The position markers 126a, 126b, 128 are configured to measure a position of the respective radiation source 104, detector 106 and subject 102. In one embodiment, the position markers 126a, 126b, 128 transmit data to the controller 120 to convey the measured position of each of the radiation source 104, detector 106 and subject 102. The controller 120 uses this data to detect any relative movement between the subject 102 and either of the radiation source 104 and the detector 106. In an example embodiment, the position markers are optical trackers which reflect light from an emitter (not shown) and the reflected light is picked up by a detector (not shown) which assesses the position of each marker based on the detected signal from each marker.

The system 100 is operated using the controller 120 which transmits and receives one or more signals with one or more components of the system 100. As shown in FIG. 1A, in one embodiment the controller 120 is communicatively coupled (e.g. wired or wireless) with each of the motors 122a, 122b, the motors 124a, 124b, the position markers 126a, 126b, 128, the radiation source 104 and the detector 106. In an embodiment, the controller 120 transmits signals to the motors 122a, 122b to move one or more of the radiation source 104 and detector 106 along one or more of the translation axes previously discussed. In an embodiment, the controller 120 transmits signals to the motors 124a, 124b to rotate one or more of the radiation source 104 and detector 106 about the rotational axes 132, 134. In an embodiment, the controller 120 receives signals from the position markers 126a, 126b, 128 to determine the position of each of the radiation source 104, detector 106 and subject 102 for purposes of detecting any relative motion therebetween. In an embodiment, the controller 120 transmits signals to the radiation source 104 to activate and deactivate the radiation fan beam 105a and receives signals from the detector 106 based on the detected radiation fan beam 105b transmitted through the subject 102.

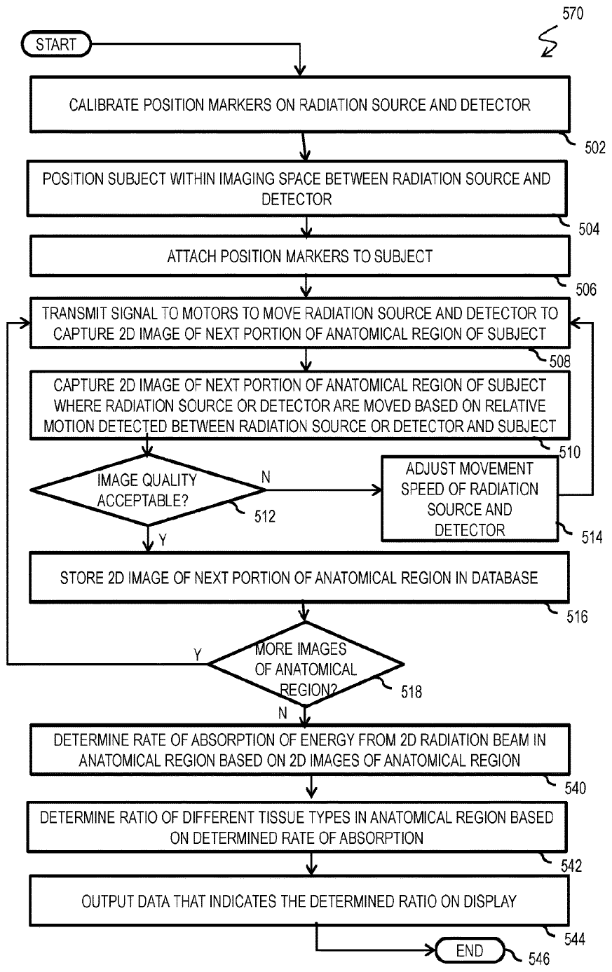

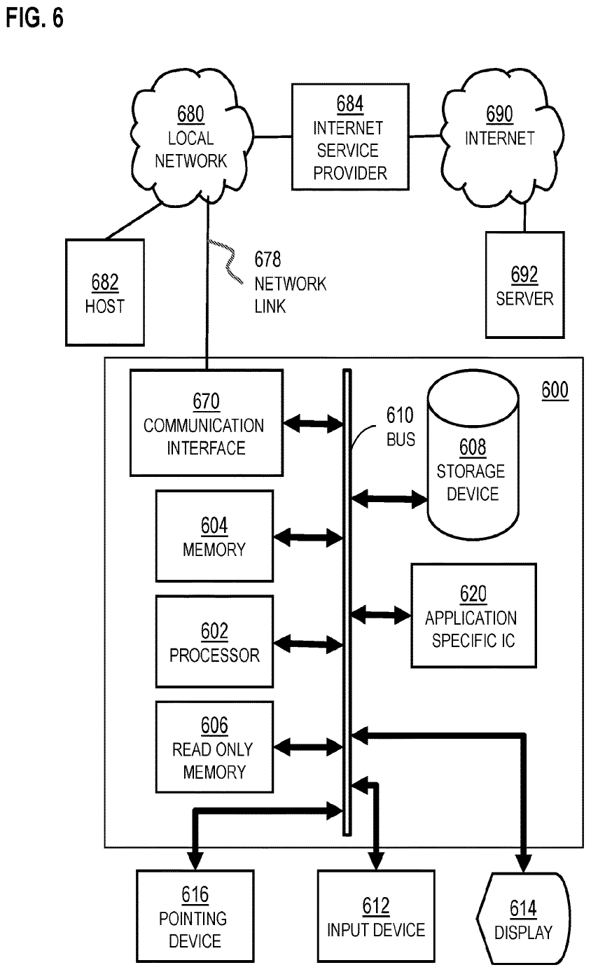

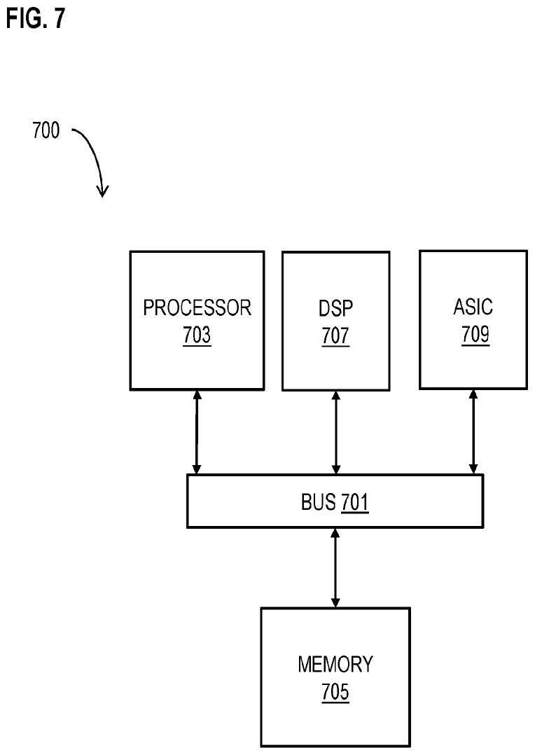

In various embodiments, the controller 120 includes an image gathering module 190 that includes instructions to cause the controller 120 to perform one or more steps of the method 500 of FIG. 5A. In other embodiments, the controller 120 includes a guided instrument module 192 that includes instructions to cause the controller 120 to perform one or more steps of the method 550 of FIG. 5B. In various embodiments, the controller 120 includes a tissue type measuring module 194 that includes instructions to cause the controller 120 to perform one or more steps of the method 570 of FIG. SC. In still other embodiments, the controller 120 is a general purpose computer system, as depicted in FIG. 6 or one or more chip sets as depicted in FIG. 7.

### 2. Method of Gathering Image Data

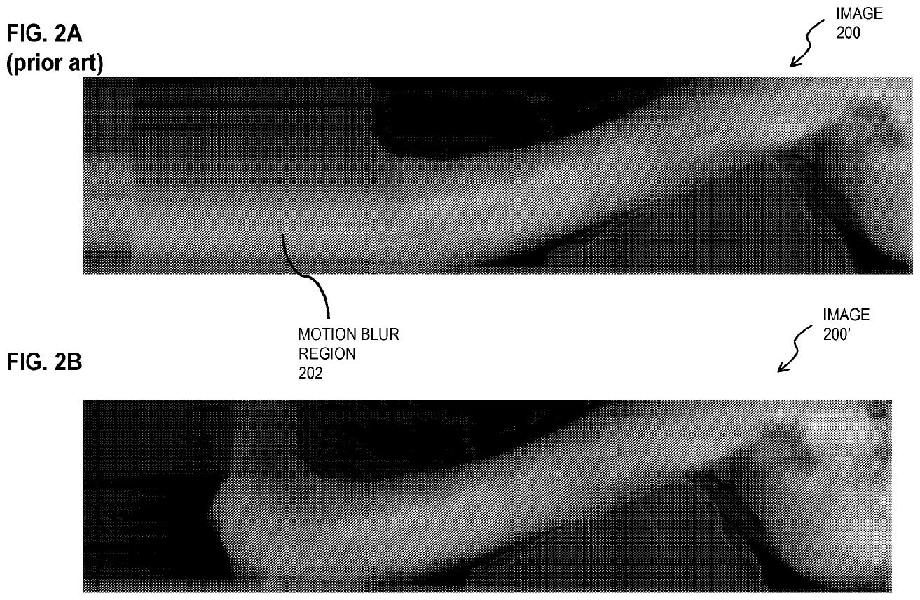

Various types of techniques are now discussed that are employed during the gathering of image data of the subject 102 with the system 100. These techniques include accounting for relative motion between the subject 102 and either the radiation source 104 and the detector 106. FIG. 2A is an image 200 that illustrates an example of image data with motion blur captured with a conventional radiographic system. As shown in FIG. 2A, the image 200 includes a motion blur region 202 where the image pixel intensity is washed out and not continuous with adjacent image regions. These motion blur regions 202 are due to relative motion between the subject and either the radiation source or detector during image capture. Conventional radiographic systems do not account for such relative motion and thus these motion blur regions 202 are common in images 200 captured with such systems. As appreciated by one of ordinary skill in the art, such motion blur regions 202 are not desirable as they cannot provide useful information regarding the anatomical region of the subject being imaged.

The inventors developed the improved system 100 with the position markers 126a, 126b, 128 that provide position data of the respective radiation source 104, detector 106 and subject 102 to the controller 120. The controller 120 can then assess whether any relative motion occurred between the subject 102 and one of the radiation source 104 and the detector 106. Upon detection of any such relative motion, the controller 120 transmits one or more signals to one of the motors 122a, 122b or motors 124a, 124b such that the radiation source 104 and/or the detector 106 are moved to offset such relative motion. The inventors recognized that this advantageously maintains the anatomical region of the subject 102 in focus during image capture. The result is the improved image 200′ shown in FIG. 2B where the motion blur region 202 is not present.

In addition to the motion correction technique, another technique that can be performed during image gathering with the system 100 is dynamic range correction.

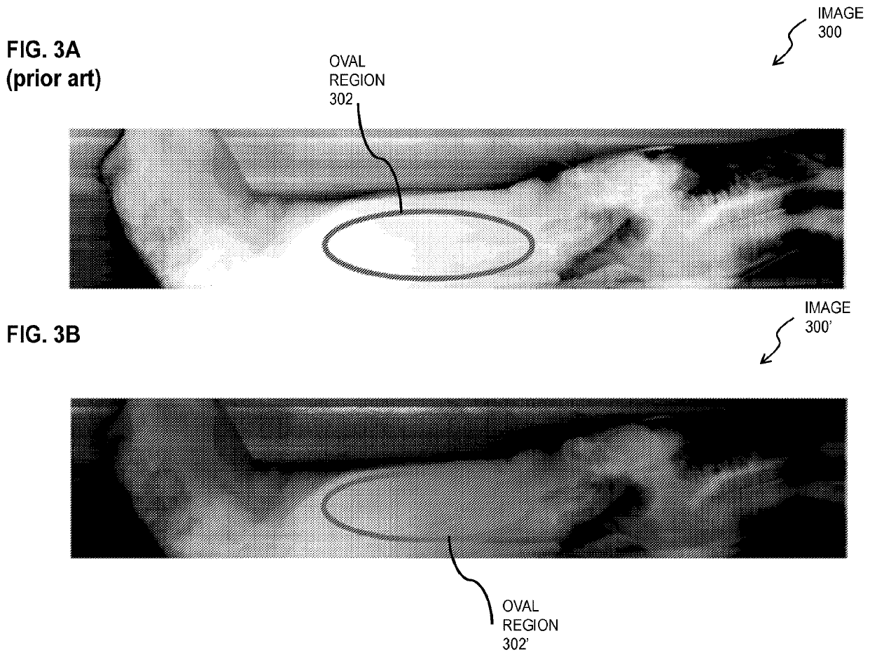

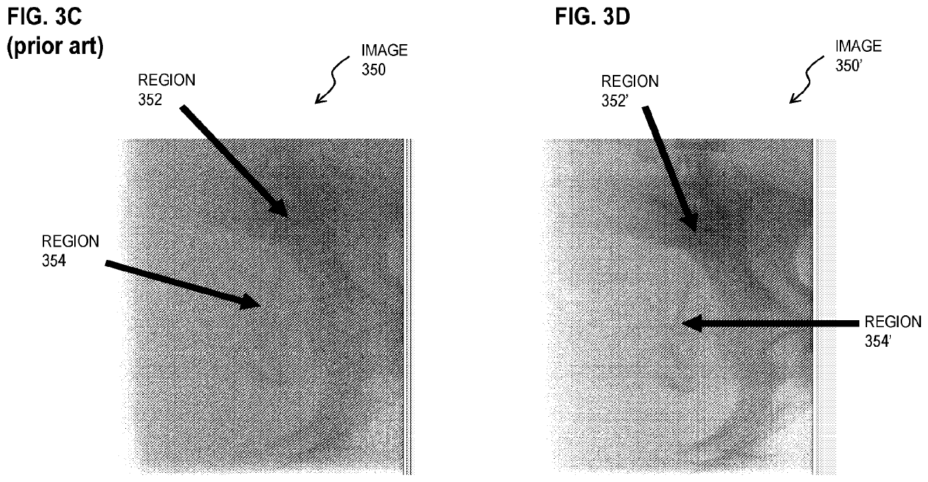

When capturing image data of the large subject 102, the radiation source 104 and the detector 106 are moved to different incremental positions along the large subject 102 (e.g., along the length 142). Image data is captured at each incremental position. The dynamic range is a range of the intensity of pixels in a captured image. FIG. 3A is an image that illustrates an example of image data captured with a conventional radiographic system having low dynamic range. As shown in the image 300 of FIG. 3A, the region 302 includes adjacent pixels with similar intensity and thus the dynamic range of this region is low. Additionally, the intensity value of these adjacent pixels is high and thus the image cannot distinguish subregions within the region 302.

In order to correct for this low dynamic range in image data captured with conventional systems, the inventors of the present invention realized that the speed of movement of the radiation source 104 and detector 106 should be adjusted. In one example embodiment, where the dynamic range is low and the pixel intensity value is high, as in the image 300 of FIG. 3A, the inventors recognized that an excess number of photons are being detected at these incremental positions along the subject. The inventors then concluded that the speed of movement of the radiation source 104 and detector 106 should be increased at these incremental positions. Increasing the speed of movement of the radiation source 104 and detector 106 would result in fewer photons being received by the detector 106 at these incremental positions and hence improved dynamic range. The result of this dynamic range correction is shown in FIG. 3B that shows an image 300′ captured by the system 100. As shown in the image 300′, the corresponding region 302 in the conventional image 300 where the dynamic range was low and the pixel intensity was excessively high is replaced by an image region 302′ with improved dynamic range where the subregions within the region 302′ of the image 300′ can be deciphered.

Similarly, in another example embodiment, where the dynamic range is low and the intensity value is low, the inventors recognized that too few photons are being detected at these incremental positions along the subject. This is shown in the image 350 of FIG. 3C, where the regions 352, 354 of the image 350 both feature low intensity due to fewer photons from these regions 352, 354. The inventors then concluded that the speed of movement of the radiation source 104 and detector 106 should be reduced for these incremental positions. Reducing the speed of movement of the radiation source 104 and detector 106 would result in more photons being received by the detector 106 at these incremental positions and hence improved dynamic range. The result of this dynamic range correction is shown in FIG. 3D that shows an image 350′ captured by the system 100. As shown in the image 350′, the corresponding regions 352, 354 in the conventional image 350 where the dynamic range was low and the pixel intensity was excessively low is replaced by image regions 352′, 354′ in the image 350′ with improved dynamic range where the subregions within the regions 352′, 354′ of the image 350′ can be deciphered.

A method that can be used to gather image data with the system 100 of FIG. 1A will now be discussed. FIG. 5A is a flow diagram that illustrates an example method 500 to capture image data with the radiographic system 100 of FIG. 1A, according to an embodiment. Although steps are depicted in FIG. 5A, and in subsequent flowcharts of FIGS. 5B and 5C, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. The method 500 starts at step 502 where the position markers 126a, 126b on the radiation source 104 and the detector 106 are calibrated. In an embodiment, in step 502 the radiation source 104 and/or the detector 106 are moved by a known distance. The data from the position markers 126a, 126b is then evaluated to verify that it measured the correct shift in the known distance of the radiation source 104 or detector 106. In this embodiment, after step 502 the position markers 126a, 126b are calibrated to accurately measure the position of the radiation source 104 and the detector 106.

In step 504, the large subject 102 (e.g. horse, cow, etc.) is positioned within an imaging space between the radiation source 104 and the detector 106. In an embodiment, in step 504 the large subject 102 is positioned such that a particular anatomical region (e.g. heart, liver or other organ) is within the imaging space such that image data of the anatomical region is gathered by the detector 106 when the radiation source 104 is activated. Additionally, in an embodiment, in step 504 the large subject 102 is positioned to be approximately equidistant (along the width dimension) between the radiation source 104 and the detector 106. In some embodiments, in step 504 the radiation source 104 and detector 106 are activated and the large subject 102 is positioned such that the image data gathered by the detector 106 is in focus. In these embodiments, the controller 120 can determine whether the image data from the detector 106 is in focus based on various factors, such as the dynamic range of the pixel intensity and/or sharp transitions in pixel intensity between adjacent pixels in the image.

In step 506, the position markers 128 are attached to the large subject 102. In an embodiment, in step 506 the position markers 128 are attached to a sufficient number of locations on the large subject 102 in order to detect relative motion between the large subject 102 and the radiation source 104 or detector 106. More specifically, in step 506 the position markers 128 are positioned at a sufficient number of locations on the large subject 102 in order to detect relative motion between the anatomical region of the subject 102 being imaged and the radiation source 104 or detector 106. Although FIG. 1A depicts four position markers 128 on the external surface of the subject 102, in other embodiments less or more than four position markers 128 may be used and may be positioned along internal surfaces of the large subject 102 (e.g. within the anatomical region being imaged). In some embodiments, in step 506 the position markers 128 are calibrated in a similar manner as the position markers 126a, 126b in step 502. In an example embodiment, in step 506 the large subject 102 is moved some known distance and the data from the position markers 128 is assessed to ensure that relative motion by this known distance is detected relative to the radiation source 104 and detector 106.

In step 508, the radiation source 104 and the detector 106 are moved such that they are positioned to capture image data from a first (or next) incremental position of an anatomical region along the large subject 102. In an embodiment, in step 508 the controller 120 transmits signals to the motors 122a and/or 124a to move the radiation source 104 and the motors 122b and/or 124b to move the detector 106 to the first incremental position. In an example embodiment, the first incremental position is a first position along the length 142 of the large subject 102 that encompasses the anatomical region being imaged. In these embodiments, a plurality of incremental positions along the subject 102 that encompass the anatomical region being imaged are stored in the memory and are retrieved by the controller 120 when performing step 508. In some embodiments, in step 508 the user uses an input device 612 (e.g. mouse, keyboard, touchscreen, etc.) (FIG. 6) of the controller 120 to select one of a plurality of anatomical regions of the subject 102 to be imaged. Based on this input, in step 508 the controller 120 transmits the signal to the motors of the system 100 to move the radiation source 104 and detector 106 to the first incremental position encompassing the anatomical region. In some embodiments, step 508 is repeated for each incremental position along the anatomical region being imaged.

In step 510, the radiation source 104 transmits the radiation fan beam 105a (e.g. x-ray fan beam) and the detector 106 captures the radiation fan beam 105b (e.g. transmitted x-ray fan beam) having transmitted through the anatomical region of the large subject 102. In an embodiment, during step 510 the controller 120 detects any relative motion between the subject 102 and the radiation source 104 or detector 106 based on data received from the position markers 126a, 126b, 128. If the controller 120 detects any such relative motion, in step 510 the controller 120 transmits signals to the motors 122a, 122b, 124a, 124b to move the radiation source 104 or detector 160 and offset such relative motion. The inventors of the present invention recognized that this advantageously ensures that the image data captured in step 510 remains in focus.

In step 512, a determination is made whether the image data captured in step 510 is acceptable. In an embodiment, in step 512 the determination is made based on whether a motion blur region 202 (FIG. 2A) is present in the image data due to relative motion that was not offset in step 510. In this embodiment, when such motion blur is detected step 510 is repeated at the same incremental position along the anatomical region in order to capture acceptable image data at this location along the large subject 102. In another embodiment, in step 512 the dynamic range of the image data captured in step 510 is assessed. In these embodiments, if the dynamic range is too low (e.g. pixel intensity range is too low) in one or more regions of the image then the determination in step 512 is that the image data is not acceptable. Additionally, in step 512 if the dynamic range is too low a further determination is made regarding whether the pixel intensity is above a threshold value, based on an excess number of photons received in step 510. Additionally, in step 512 if the dynamic range is too low a further determination is made regarding whether the pixel intensity is below a threshold value, based on an insufficient number of photons received in step 510.

In step 514, if the determination in step 512 is in the negative and the pixel intensity is above a threshold value, the speed of movement of the radiation source 104 and detector 106 is increased. In this embodiment, in step 514 the controller 120 transmits signals to the motors 122 or 124 to increase the speed at which the radiation source 104 or detector 106 are moved along the incremental positions of the anatomical region of the subject 102. The inventors recognized that this advantageously reduces the number of photons received by the detector 106 during step 510 at each incremental position. Hence, the dynamic range of the image data gathered in step 510 is enhanced. Similarly, if the determination in step 512 is in the negative and the pixel intensity is below a threshold value, the speed of movement of the radiation source 104 and detector 106 is decreased. In this embodiment, in step 514 the controller 120 transmits signals to the motors 122 or 124 to reduce the speed at which the radiation source 104 or detector 106 are moved along the incremental positions of the anatomical region of the subject 102. The inventors recognized that this advantageously increases the number of photons received by the detector 106 during step 510 at each incremental position. Hence, the dynamic range of the image data gathered in step 510 is enhanced.

In step 516, if the determination in step 512 is in the affirmative then the method 500 moves to block 516 where the image data captured in step 510 is stored in a memory of the controller 120.

In step 518, a determination is made as to whether additional incremental positions along the anatomical region of the large subject 102 are to be imaged. This step 518 is based on whether the radiation source 104 and detector 106 have moved across each of the incremental positions along the anatomical region of the large subject 102. If this determination is in the negative then image data at each incremental position has been captured and thus the method 500 ends at block 520. If this determination is in the affirmative, then image data has yet to be captured at one or more incremental positions along the anatomical region of the large subject 102 and thus the method 500 moves back to step 508. Steps 508 through 518 are then repeated for each incremental position along the anatomical region yet to be imaged.

### 3. Method of Providing Treatment With Guided Instrument

In some embodiments, the image data captured with the system 100 can be used for various applications. One such application is treatment of the anatomical region of the large subject 102 that was imaged by the system 100. In some embodiments, the system 100 includes a guided instrument 130 that can be used to perform these treatments. As shown in FIG. 1A, in these embodiments the guided instrument 130 can include a position marker 129 that is similar to the position markers 126a, 126b, 128 and is used to provide position data of the guided instrument 130 to the controller 120.

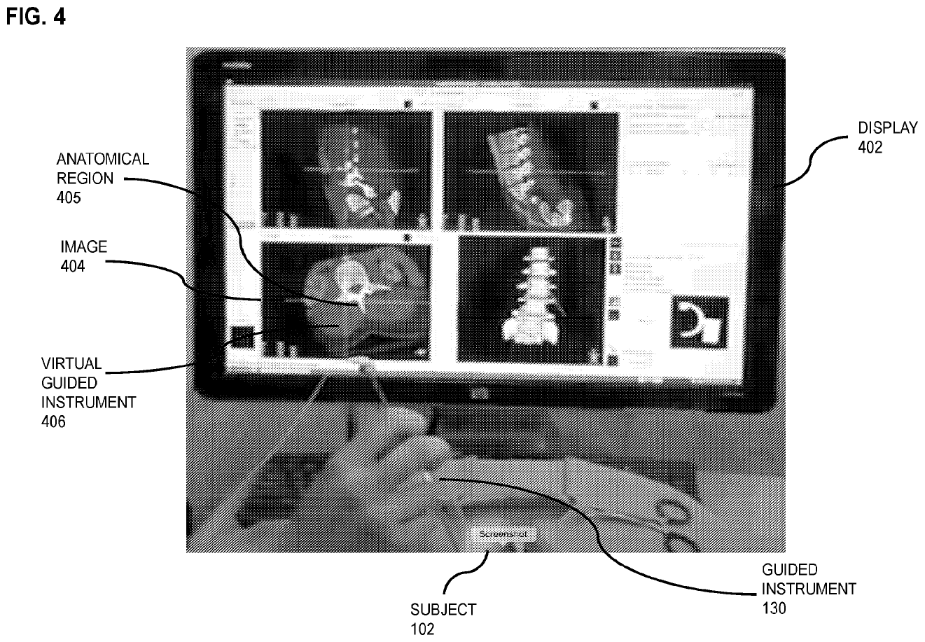

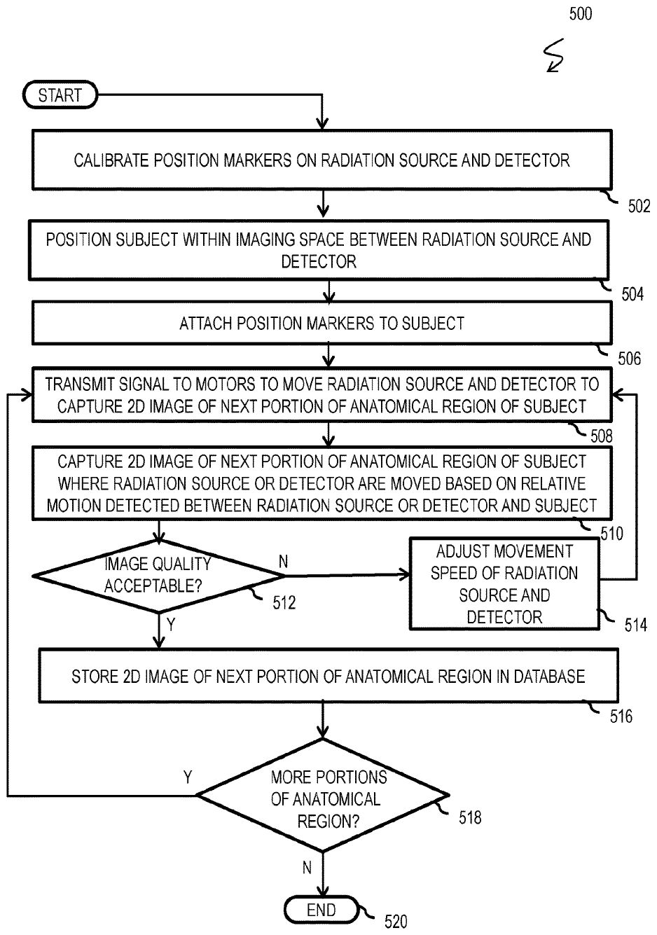

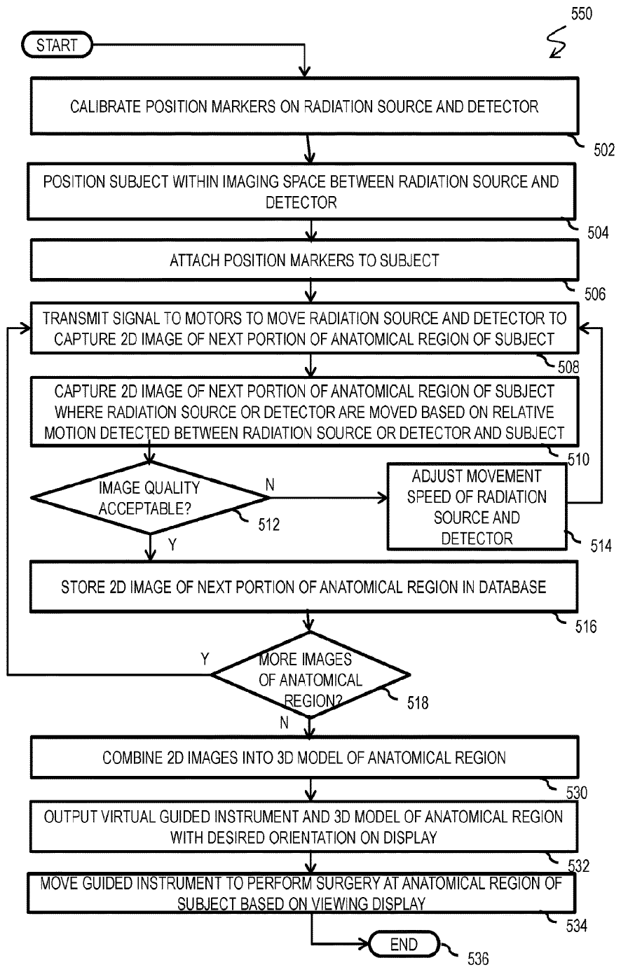

FIG. 4 is an image that illustrates an example of a front view of a display 402 used while performing a treatment of an anatomical region of the large subject 102, according to an embodiment. As shown in FIG. 4, the guided instrument 130 is provided and is moved by a medical professional (e.g. veterinary surgeon) to perform the treatment on the anatomical region of the large subject 102. As shown in FIG. 4, the display 402 outputs an image 404 that was captured by the system 100 of the anatomical region 405 of the large subject 102. In some embodiments, the image 404 that is output on the display 402 combines image data captured at multiple incremental positions along the subject 102 which encompass the anatomical region 405. In one embodiment, the user of the system 100 (e.g. medical professional performing the treatment) can adjust an orientation of the image 404 on the display 402. As further shown in FIG. 4, the display 402 also outputs a virtual guided instrument 406 that indicates the position of the guided instrument 130 relative to the anatomical region 405. The position data from the position marker 129 on the guided instrument 130 is provided to the controller 120 which then communicates with the display 402 to output the virtual guided instrument 406 on the display 402. In these embodiments, as the medical professional observers the display 402, one or more steps of the medical treatment are performed. The simultaneous output of the virtual guided instrument 406 and the image 404 of the anatomical region 405 of the large subject 102 on the display 402 advantageously permits the medical professional to perform the treatment while observing the display 402.

In one embodiment, the treatments and corresponding anatomical regions that can be performed include but are not limited to dorsal spinous process resection (spine), standing endoscopic vertebral foraminotomy for nerve root compression (spine) and guidance for injections (e.g. any joint as recognized by one of ordinary skill in the art). Additionally, there are many other therapeutic approaches, such as those focused on the head, neck and spine. However, the treatments and corresponding anatomical regions are not limited to any particular treatment of any specific anatomical region and encompasses any such treatment and anatomical region recognized by one of ordinary skill in the art. In another embodiment, the treatments and corresponding anatomical regions will depend on the category of the large subject (e.g. horse, cow, dog, human, dolphin, etc.) A method for performing treatment of the large subject with the guided instrument will now be discussed. FIG. 5B is a flow diagram that illustrates an example method 550 to perform treatment on the large subject 102 with captured image data from the radiographic system 100 of FIG. 1A, according to an embodiment. Steps 502 through 518 of the method 550 are the same as steps 502 through 518 of the method 500 previously discussed. In an embodiment, steps 502 through 518 are performed to gather image data of the anatomical region of the large subject 102 at a plurality of incremental positions. However, in some embodiments the method 550 need not perform steps 502 through 518. In these embodiments, the method 550 merely receives the image data that was gathered by the method 500 and uses this image data to perform the treatment of the large subject. In these embodiments, steps 502 through 518 are omitted.

In step 530, the image data obtained in step 516 at one or more incremental positions along the anatomical region of the subject 102 are combined. In an embodiment, in step 530 a 3D model is obtained of the anatomical region based on this combination. In some embodiments, in step 530 the image data captured at a small number of incremental positions (e.g. less than 10) is combined in order to enhance computational efficiency. As appreciated by one of ordinary skill in the art, combining a large number of 2D image data into a 3D model can reduce computational efficiency. Thus, the inventors of the present invention recognized that it would be advantageous in step 530 to only combine image data captured in step 516 from those incremental positions along the anatomical region where the treatment is to be performed (or from where the medical professional would need to view when performing said treatment).

In step 532, a virtual guided instrument is output on a display 402, 614 along with the 3D model obtained from step 530. In an embodiment, the image 404 output on the display 402 of FIG. 4 is the 3D model of the anatomical region 405. In an embodiment, in step 532 the 3D model obtained in step 530 is output on the display 402, 614 after which the medical professional can adjust the orientation of the 3D model (e.g. with the input device 612, such as a mouse, keyboard or touchscreen) to a desired orientation. In an example embodiment, the desired orientation is that orientation which corresponds to a first person view (e.g. front view) of the anatomical region 405 from the perspective of the medical professional.

In another embodiment, in step 532 the virtual guided instrument 406 is also output on the display 402, 614 such that the virtual guide instrument 406 overlays the anatomical region 405 of the image 404. As previously disclosed, as the medical professional moves the guided instrument 130 to perform the treatment at the anatomical region 405 of the subject 102, the controller 120 receives position information of the guided instrument 130 from the position tracker 129 and moves the virtual guided instrument 406 on the display 402 in real time based on this position information.

In step 534, the medical professional moves the guided instrument 130 to perform the treatment on the anatomical region 405 of the subject 102. In an embodiment, in step 534 as the medical professional moves the guided instrument 130, the controller 120 varies the position of the virtual guided instrument 406 on the display 402. Thus, in step 534 the medical professional performs the treatment while observing the display 402. In an example embodiment, the medical professional performs the treatment in step 534 as the image 404 is oriented at a desired orientation on the display 402 (from step 532).

Although step 534 is discussed in the context of the medical professional moving the guided instrument 130, in other embodiments the guided instrument 130 is moved by an automatic means (e.g. robotic arm) attached to the guided instrument 130. In these embodiments, in step 534 the controller 120 determines a trajectory of movement of the guided instrument 130 to perform the treatment. The controller 120 then transmits position data of this determined trajectory to the robotic arm that automatically moves the guided instrument 130 to perform the treatment. In still other embodiments, in step 534 the medical professional moves an input device that is remotely attached to the guided instrument 130 such that the medical professional moves the guided instrument 130 via. movement of the input device. One advantage of this latter embodiment is that the medical professional can perform the treatment outside the radiation field between the radiation source 104 and the detector 106. Conventional systems involve multiple medical professionals in the room housing the system who perform various actions, such as holding equipment and positioning the subject. Since the system disclosed herein uses robotic control, only one person is positioned in the room housing the system as opposed to multiple (e.g. three) in conventional systems. Consequently, in these embodiments, the total radiation field that is experienced by humans operating the system is reduced by a factor of three. In some embodiments, the medical professional in the system disclosed herein is positioned proximate to the head of the subject and thus not proximate to the area of the subject that is being imaged. This further reduces the radiation exposure of medical professionals, as compared to conventional systems, by an additional factor of about two. Additionally, in these embodiments, since the medical professional of the system disclosed herein is not positioned proximate to the radiation source, the system provides a greater opportunity to use shielding between the subject and the medical professional which further reduces the radiation exposure by a factor of between about three and about four. Thus, in these embodiments, due to the reduction in the number of medical professionals, the remote positioning of the medical professionals and the introduction of the shielding barrier, the system disclosed herein can reduce the radiation exposure by a factor between about 5 and about 10.

In some embodiments, the method 550 including steps 530 through 534 are performed on a horse subject 102. In these embodiments, steps 530 through 534 are performed without administering anesthesia to the horse subject 102. Additionally, in these embodiments, the horse subject 102 remains standing as steps 530 through 534 are performed. The inventors of the present invention recognized that this advantageously eliminates several steps that are routinely required during conventional equine medical procedures (e.g. anesthesia, laying the horse on a flat surface, etc.).

In some embodiments, during the steps 530 through 534 the controller 120 receives position data from the position trackers 128, 129 to detect relative motion between the subject 102 and the guided instrument 130. The controller 120 then transmits a signal to automatically move the position of the guided instrument 130 to offset such relative motion during steps 530 through 534. In an embodiment, during steps 530 through 534, the system will move to maintain a fixed relationship between the guided instrument 130 and the moving subject 102 for the purposes of imaging. Once the images are captured, then the guided instrument 130 can be moved relative to the animal (e.g., using dynamic references on the subject and the instrument) without any further involvement of the robotic imaging system. The inventors recognized that this advantageously prevents relative motion between the subject 102 and the guided instrument 130 from affecting the treatment.

### 4. Method for Measuring a Ratio of Tissue Types in a Large Subject

In some embodiments, the image data captured with the system 100 can be used for various applications. One such application is to measure a ratio of one or more tissue types (e.g. fat %) within an anatomical region of the large subject 102. In one example embodiment, the percentage of fat can be measured in an anatomical region of a cow subject. The inventors of the present invention recognized that the image data gathered by the system 100 could be used to measure this ratio of one or more tissue types anti-mortem, The inventors recognized that the ratio of these tissue types is routinely measured post-mortem and that measuring this ratio anti-mortem would provide valuable information to various industries, such as meat industries and farm industries for various applications (e.g. establishing best practices for raising cattle for high quality meat production, etc.).

A method for measuring the ratio of one or more tissue types within the anatomical region of the large subject will now be discussed. FIG. 5C is a flow diagram that illustrates an example method 570 to measure this ratio with captured image data from the radiographic system 100 of FIG. 1A, according to an embodiment. Steps 502 through 518 of the method 570 are the same as steps 502 through 518 of the method 500 previously discussed. In an embodiment, steps 502 through 518 are performed to gather image data of the anatomical region of the large subject 102 at a plurality of incremental positions. However, in some embodiments the method 570 need not perform steps 502 through 518. In these embodiments, the method 570 merely receives the image data that was gathered by the method 500 and uses this image data to measure the ratio of one or more tissue types in the large subject. In these embodiments, steps 502 through 518 are omitted.

In step 540, a rate of absorption of one or more energies of the radiation fan beam 105a in the anatomical region of the large subject 102 is determined. In an embodiment, the rate of energy absorption at each incremental position along the anatomical region of the large subject 102 is determined based on the image data gathered in step 516.

In step 542, the ratio of the one or more different tissue types in the anatomical region of the large subject 102 is determined. In an embodiment, the ratio of the one or more tissue types (e.g. percentage of fat) is determined based on the determined rate of energy absorption in the anatomical region from step 540.

In step 544, output data is provided on a display to indicate the ratio of the one or more different tissue types determined in step 542. In an embodiment, in step 544 the value of the determined ratio (e.g. the value of the % fat in the anatomical region of the subject 102) is output on the display 402, 614. However, in other embodiments the value of the determined ratio need not be output on a display and instead is stored in a memory of the controller 120 or transmitted to a processor or controller at a remote location.

In an embodiment, the steps 540 through 544 are performed anti-mortem. In an example embodiment, measuring the ratio of one or more tissue types anti-mortem is advantageous over conventional methods which measure the ratio of the one or more tissue types post-mortem. The inventors recognized that measuring the ratio of the one or more tissue types anti-mortem is more accurate than measuring the ratio post-mortem. The inventors recognized various advantages of measuring the ratio anti-mortem. For example, when the ratio is measured anti-mortem, the diet or feeding regimen of the subject can be adjusted to achieve a desired ratio (e.g. desired fat/lean composition). Another advantage is that the measured ratio anti-mortem can assist with optimizing farming of the large subjects (e.g. to enhance the efficiency and/or profitability of the farm).

In an embodiment, the steps 540 through 544 are performed using the system 100 where the radiation source 104 and detector 106 are held in a fixed position and one or more large subjects 102 (e.g. cows) are moved through the imaging space between the radiation source 104 and the detector 106. As each subject 102 (e.g. cow) is walked through the imaging space, the controller 120 performs steps 540 through 544 and outputs or stores the measured ratio of the one or more tissue types. In these embodiments, the controller 120 can also store an identifier (e.g. number) for each large subject 102 (e.g. cow) to correlate the determined ratio of the tissue type with each large subject 102. In an embodiment, this data can then be used to various industries (e.g. meat industry to assess meat quality, farming industry to assess best practices for farming in order to raise cows for quality meat production, etc.).

### 5. Hardware Overview

FIG. 6 is a block diagram that illustrates a computer system 600 upon which an embodiment of the invention may be implemented. Computer system 600 includes a communication mechanism such as a bus 610 for passing information between other internal and external components of the computer system 600. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number of code for a character. In some embodiments, information called analog data is represented by a near continuum of measurable values within a particular range. Computer system 600, or a portion thereof, constitutes a means for performing one or more steps of one or more methods described herein.

A sequence of binary digits constitutes digital data that is used to represent a number or code for a character. A bus 610 includes many parallel conductors of information so that information is transferred quickly among devices coupled to the bus 610, One or more processors 602 for processing information are coupled with the bus 610. A processor 602 performs a set of operations on information. The set of operations include bringing information in from the bus 610 and placing information on the bus 610. The set of operations also typically include comparing two or more units of information, shifting positions of units of information, and combining two or more units of information, such as by addition or multiplication. A sequence of operations to be executed by the processor 602 constitutes computer instructions.

Computer system 600 also includes a memory 604 coupled to bus 610. The memory 604, such as a random access memory (RAM) or other dynamic storage device, stores information including computer instructions. Dynamic memory allows information stored therein to be changed by the computer system 600. RAM allows a unit of information stored at a location called a memory address to be stored and retrieved independently of information at neighboring addresses. The memory 604 is also used by the processor 602 to store temporary values during execution of computer instructions. The computer system 600 also includes a read only memory (ROM) 606 or other static storage device coupled to the bus 610 for storing static information, including instructions, that is not changed by the computer system 600. Also coupled to bus 610 is a non-volatile (persistent) storage device 608, such as a magnetic disk or optical disk, for storing information, including instructions, that persists even when the computer system 600 is turned off or otherwise loses power.

Information, including instructions, is provided to the bus 610 for use by the processor from an external input device 612, such as a keyboard containing alphanumeric keys operated by a human user, or a sensor. A sensor detects conditions in its vicinity and transforms those detections into signals compatible with the signals used to represent information in computer system 600. Other external devices coupled to bus 610, used primarily for interacting with humans, include a display device 614, such as a cathode ray tube (CRT) or a liquid crystal display (LCD), for presenting images, and a pointing device 616, such as a mouse or a trackball or cursor direction keys, for controlling a position of a small cursor image presented on the display 614 and issuing commands associated with graphical elements presented on the display 614.

In the illustrated embodiment, special purpose hardware, such as an application specific integrated circuit (IC) 620, is coupled to bus 610. The special purpose hardware is configured to perform operations not performed by processor 602 quickly enough for special purposes. Examples of application specific ICs include graphics accelerator cards for generating images for display 614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 600 also includes one or more instances of a communications interface 670 coupled to bus 610. Communication interface 670 provides a two-way communication coupling to a variety of external devices that operate with their own processors, such as printers, scanners and external disks. In general the coupling is with a network link 678 that is connected to a local network 680 to which a variety of external devices with their own processors are connected. For example, communication interface 670 may be a parallel port or a serial port or a universal serial bus (USB) port on a personal computer. In some embodiments, communications interface 670 is an integrated services digital network (ISDN) card or a digital subscriber line (DSL) card or a telephone modem that provides an information communication connection to a corresponding type of telephone line. In some embodiments, a communication interface 670 is a cable modem that converts signals on bus 610 into signals for a communication connection over a coaxial cable or into optical signals for a communication connection over a fiber optic cable. As another example, communications interface 670 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 670 sends and receives electrical, acoustic or electromagnetic signals, including infrared and optical signals, that carry information streams, such as digital data.

The term computer-readable medium is used herein to refer to any medium that participates in providing information to processor 602, including instructions for execution. Such a medium may take many forms, including, but not limited to, non-volatile media, volatile media and transmission media. Non-volatile media include, for example, optical or magnetic disks, such as storage device 608. Volatile media include, for example, dynamic memory 604. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 602, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 602, except for carrier waves and other signals.

Logic encoded in one or more tangible media includes one or both of processor instructions on a computer-readable storage media and special purpose hardware, such as ASIC *620.

Network link 678 typically provides information communication through one or more networks to other devices that use or process the information. For example, network link 678 may provide a connection through local network 680 to a host computer 682 or to equipment 684 operated by an Internet Service Provider (ISP). ISP equipment 684 in turn provides data communication services through the public, world-wide packet-switching communication network of networks now commonly referred to as the Internet 690. A computer called a server 692 connected to the Internet provides a service in response to information received over the Internet. For example, server 692 provides information representing video data for presentation at display 614.

The invention is related to the use of computer system 600 for implementing the techniques described herein. According to one embodiment of the invention, those techniques are performed by computer system 600 in response to processor 602 executing one or more sequences of one or more instructions contained in memory 604. Such instructions, also called software and program code, may be read into memory 604 from another computer-readable medium such as storage device 608. Execution of the sequences of instructions contained in memory 604 causes processor 602 to perform the method steps described herein. In alternative embodiments, hardware, such as application specific integrated circuit 620, may be used in place of or in combination with software to implement the invention. Thus, embodiments of the invention are not limited to any specific combination of hardware and software.

The signals transmitted over network link 678 and other networks through communications interface 670, carry information to and from computer system 600. Computer system 600 can send and receive information, including program code, through the networks 680, 690 among others, through network link 678 and communications interface 670. In an example using the Internet 690, a server 692 transmits program code for a particular application, requested by a message sent from computer 600, through Internet 690, ISP equipment 684, local network 680 and communications interface 670. The received code may be executed by processor 602 as it is received, or may be stored in storage device 608 or other non-volatile storage for later execution, or both. In this manner, computer system 600 may obtain application program code in the form of a signal on a carrier wave.

Various forms of computer readable media may be involved in carrying one or more sequence of instructions or data or both to processor 602 for execution. For example, instructions and data may initially be carried on a magnetic disk of a remote computer such as host 682. The remote computer loads the instructions and data into its dynamic memory and sends the instructions and data over a telephone line using a modem. A modem local to the computer system 600 receives the instructions and data on a telephone line and uses an infra-red transmitter to convert the instructions and data to a signal on an infra-red a carrier wave serving as the network link 678. An infrared detector serving as communications interface 670 receives the instructions and data carried in the infrared signal and places information representing the instructions and data onto bus 610. Bus 610 carries the information to memory 604 from which processor 602 retrieves and executes the instructions using some of the data sent with the instructions. The instructions and data received in memory 604 may optionally be stored on storage device 608, either before or after execution by the processor 602.

FIG. 7 illustrates a chip set 700 upon which an embodiment of the invention may be implemented. Chip set 700 is programmed to perform one or more steps of a method described herein and includes, for instance, the processor and memory components described with respect to FIG. 6 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 700, or a portion thereof, constitutes a means for performing one or more steps of a method described herein.

In one embodiment, the chip set 700 includes a communication mechanism such as a bus 701 for passing information among the components of the chip set 700. A processor 703 has connectivity to the bus 701 to execute instructions and process information stored in, for example, a memory 705. The processor 703 may include one or more processing cores with each core configured to perform independently. A multi-core processor enables multiprocessing within a single physical package. Examples of a multi-core processor include two, four, eight, or greater numbers of processing cores. Alternatively or in addition, the processor 703 may include one or more microprocessors configured in tandem via the bus 701 to enable independent execution of instructions, pipelining, and multithreading. The processor 703 may also be accompanied with one or more specialized components to perform certain processing functions and tasks such as one or more digital signal processors (DSP) 707, or one or more application-specific integrated circuits (ASIC) 709. A DSP 707 typically is configured to process real-world signals (e.g., sound) in real time independently of the processor 703. Similarly, an ASIC 709 can be configured to performed specialized functions not easily performed by a general purposed processor, Other specialized components to aid in performing the inventive functions described herein include one or more field programmable gate arrays (FPGA) (not shown), one or more controllers (not shown), or one or more other special-purpose computer chips.

The processor 703 and accompanying components have connectivity to the memory 705 via the bus 701. The memory 705 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 705 also stores the data associated with or generated by the execution of one or more steps of the methods described herein.

### 6. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

What is claimed is:

1. A system comprising:
a radiation source configured to generate a radiation fan beam in a two-dimensional (2D) plane;
a detector spaced apart from the radiation source and configured to detect the radiation fan beam in the 2D plane;
a first support configured to mount the radiation source, said first support configured to translate the radiation source along a first axis and further configured to rotate the radiation source relative to the first axis;
a second support configured to mount the detector, said second support configured to translate the detector along a second axis and further configured to rotate the radiation source relative to the second plane;
a position marker coupled to each of the radiation source, the detector and a subject positioned between the radiation source and the detector, said position marker configured to measure a position of each of the radiation source, the detector and the subject;
at least one processor; and
at least one memory including one or more sequences of instructions,
the at least one memory and the one or more sequences of instructions configured to, with the at least one processor, cause the system to perform at least the following,
transmit a first signal to the first support and the second support to cause the radiation source and the detector to move to each of a plurality of incremental positions along an anatomical region of the subject;
receive motion data, at each incremental position, from the position marker coupled to each of the radiation source, the detector and the subject to detect relative motion between the subject and the radiation source or the detector;
transmit a second signal to the first support and the second support, at each incremental position, to move the radiation source and the detector to offset the detected relative motion;
transmit a third signal to the radiation source to cause the radiation source to generate the radiation fan beam in the 2D plane at each incremental position along the anatomical region of the subject;
receive image data from the detector based on the detection of the radiation fan beam in the 2D plane at each incremental position; and
store, in the memory, the image data at each incremental position.

2. The system of claim 1,
wherein the radiation source is an x-ray tube and wherein the radiation fan beam is an x-ray fan beam in the 2D plane; and
wherein the detector is an x-ray detector comprising a linear array of detectors configured to detect the x-ray fan beam in the 2D plane.

3. The system of claim 1, wherein the first support comprises:
a first gantry horizontal support configured to translate the radiation source along the first axis to vary the spacing between the radiation source and the detector, and
a first gantry vertical support configured to translate the radiation source in a first plane that is orthogonal to the first axis.

4. The system of claim 3, wherein the second support comprises:
a second gantry horizontal support configured to translate the detector along the second axis to vary the spacing between the radiation source and the detector; and
a second gantry vertical support configured to translate the detector in a second plane that is orthogonal to the second axis.

5. The system of claim 4, further comprising:
a first wrist configured to mount the radiation source to the first gantry horizontal support, said first wrist configured to rotate the radiation source relative to the first axis; and
a second wrist configured to mount the detector to the second gantry horizontal support, said second wrist configured to rotate the detector relative to the second axis.

6. The system of claim 4,
wherein the first gantry vertical support is configured to translate the radiation source in the first plane to encompass a length of the subject and a height of the subject; and
wherein the second gantry vertical support is configured to translate the detector in the second plane to encompass the length of the subject and the height of the subject.

7. The system of claim 6, wherein the length of the subject is at least 6 feet and the height of the subject is at least 4 feet.

8. The system of claim 1, wherein the at least one memory and the one or more sequences of instructions are further configured to cause the system to:
determine a dynamic range of pixel intensity of the image data received from the detector at each incremental position; and
adjust the first signal transmitted to the first support and the second support at each incremental position to vary a speed of movement of the radiation source and the detector to a next incremental position based on the determined dynamic range.

9. The system of claim 8, wherein the at least one memory and the one or more sequences of instructions are further configured to cause the system to:
adjust the first signal transmitted to the first support and the second support to reduce the speed of movement of the radiation source and the detector to the next incremental position based on determining that the dynamic range is below a threshold range and that values of the pixel intensity within the dynamic range are below a threshold value; and
adjust the first signal transmitted to the first support and the second support to increase the speed of movement of the radiation source and the detector to the next incremental position based on determining that the dynamic range is below the threshold range and that values of the pixel intensity within the dynamic range are above the threshold value.

10. A method comprising:
transmitting, from a processor, a first signal to a first support and a second support to cause a radiation source and a detector to move to each of a plurality of incremental positions along an anatomical region of a subject;
receiving, from position markers attached to each of the radiation source, the detector and the subject, motion data to detect relative motion between the subject and the radiation source or the detector at each incremental position;
transmitting, from the processor, a second signal to the first support and the second support to move the radiation source and the detector to offset the detected relative motion at each incremental position;
transmitting, from the processor, a third signal to the radiation source to cause the radiation source to generate a radiation fan beam in a two-dimensional (2D) plane at each incremental position;
receiving, from the detector, image data based on detection of the radiation fan beam in the 2D plane at each incremental position; and
storing, in a memory, the image data at each incremental position.

11. The method of claim 10, further comprising:
determining, with the processor, a dynamic range of pixel intensity of the image data received from the detector at each incremental position, and
adjusting the transmission of the first signal to the first support and the second support at each incremental position to vary a speed of movement of the radiation source and the detector to a next incremental position based on the determined dynamic range.

12. The method of claim 11, wherein the adjusting step comprises:
adjusting the transmission of the first signal to reduce the speed of movement of the radiation source and the detector to the next incremental position based on determining that the dynamic range is below a threshold range and that values of the pixel intensity within the dynamic range are below a threshold value; and
adjusting the transmission of the first signal to increase the speed of movement of the radiation source and the detector to the next incremental position based on determining that the dynamic range is below the threshold range and that values of the pixel intensity within the dynamic range are above the threshold value.

13. The method of claim 10, further comprising:
providing a guided instrument with a position marker;
combining, with the processor, the stored image data at one or more incremental positions of the anatomical region into a 3D model of the anatomical region of the subject;
outputting, on a display, the 3D model of the anatomical region of the subject;
varying, with an input device, an orientation of the 3D model on the display to a desired orientation,
receiving, at the processor, data from the position marker of the guided instrument;
outputting, on the display, a virtual guided instrument in the desired orientation of the 3D model based on the received data from the position marker of the guided instrument; and
moving the guided instrument to perform a treatment of the anatomical region of the subject based on viewing the virtual guided instrument moving relative to the desired orientation of the 3D model on the display.

14. The method of claim 13, wherein the subject is a horse and wherein the steps of the method are performed without administering anesthesia.

15. The method of claim 14, wherein the horse is standing as the steps of the method are performed.

16. The method of claim 10, further comprising:
determining, with the processor, a rate of energy absorption of the radiation fan beam in the 2D plane in the anatomical region based on the received image data at each incremental position;
determining, with the processor, a ratio of one or more tissue types within the anatomical region of the subject based on the determined rate of absorption;
outputting, on a display, data that indicates the determined ratio of the one or more tissue types within the anatomical region of the subject.

17. The method of claim 16, wherein the ratio of the one or more tissue types is a percentage of body fat in the anatomical region of the subject.

18. The method of claim 17, wherein the subject is a cow and wherein the steps of the method are performed anti-mortem while the cow is standing.

19. A method comprising:
providing a guided instrument with a position marker,
combining, with a processor, image data captured at one or more incremental positions of an anatomical region of a subject into a 3D model of the anatomical region of the subject;
outputting, on a display, the 3D model of the anatomical region of the subject;
varying, with an input device, an orientation of the 3D model on the display to a desired orientation:
receiving, at the processor, data from the position marker of the guided instrument;
outputting, on the display, a virtual guided instrument in the desired orientation of the 3D model based on the received data from the position marker of the guided instrument; and
moving the guided instrument to perform a treatment of the anatomical region of the subject based on viewing the virtual guided instrument moving relative to the desired orientation of the 3D model on the display.

20. A method comprising:
determining, with a processor, a rate of energy absorption of a radiation fan beam in a 2D plane in an anatomical region of a subject based on image data captured at one or more incremental positions along the anatomical region of the subject;
determining, with the processor, a ratio of one or more tissue types within the anatomical region of the subject based on the determined rate of absorption; and
outputting, on a display, data that indicates the determined ratio of the one or more tissue types within the anatomical region of the subject.
